(19) United States
(12) Patent Application Publication
(10) Pub. No.: US 2003/0171393 A1
(43) Pub. Date: Sep. 11, 2003

(54) Drugs for sex dysfunctions

(76) Inventor: Piero Del Soldato, Milano (IT)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/333,927

(22) PCT Filed: Jul. 27, 2001
(86) PCT No.: PCT/EP01/08733

(30) Foreign Application Priority Data
Sep. 8, 2000 (IT) MI2000A001847

(51) Int. Cl. A61K 31/4741; A61K 31/44; A61K 31/407; A61K 31/4035; A61K 31/403; A61K 31/381; A61K 31/401; A61K 31/21

(52) U.S. Cl. 514/291; 514/509; 514/352; 514/411; 514/447; 514/450; 514/416; 514/423

(57) ABSTRACT

Use in sex dysfunctions of one or more of the following classes of drugs selected from the following: B) Salified and not salified, nitric oxide-donor drugs, of formula A—X₁—N(O)_{z}, C) Organic or inorganic salts of compounds inhibiting phospho-diesterases.

The present invention relates to drugs to be utilized for systemic and topical use in the sex dysfunction therapy, specifically in the male impotence and in female sex dysfunctions.

All over the world there is a progressive ageing of the population. It is expected that in about 5 years 17% of the population is over sixty-five. This phenomenon involves important consequences not only from a sociological point of view, but also from an epidemiological point of view. If at the beginning of the century the diseases having a greater impact on mortality and morbidity were the infectious ones, now other kinds of diseases have a greater importance. Among these, sex dysfunctions in both sexes are to be considered, which affect a very significant percentage of the population, especially due to the progressive ageing.

The male impotence or erectile dysfunction is a diffused disease. In the United States it is estimated that the impotence regards from 10 to 20 millions people over 18 years and that in the male population over forty the impotence reaches a percentage of 52%. Analogously, also a very high percentage of women (up to 76%) suffers from sex dysfunctions. For both pathologies sildenafil citrate is commonly used even though with not completely satisfactory results. The sildenafil citrate is an active drug by os exerting a beneficial vasoactive action in the male sex district. The main problem connected to the administration of this drug resides in the impossibility to dissociate its efficacy from the toxic effects, since sildenafil citrate acts strengthening the effects induced by a high production of nitric oxide, (J. Urol. 1998, 160, 257-61) and under these conditions it causes significant toxic effects. Indeed the drug is badly tolerated in patients subjected to therapy with nitrate drugs and it causes cephalea in more than 16% of the cases, so that the use is contraindicated in these therapeutic treatments. The drug is badly tolerated even when it is taken by patients affected by pathologies characterized by a high endogenous hyperproduction of nitric oxide, such as for example cardiomyopathies (J. Am. Coll. Cardiol. 29, 716-24, 1997), infarct (Am. J. Hypertens. 1, 174-182 1999), cardiac decompensation. It is indeed known that the Sildenafil citrate has caused serious, even lethal, side effects in cardiopathic patients (Am. J. Cardiol. 84/5B, 11N-17N, 1999).

From the patent application WO 99/67231 the relaxing effect on the cavernous artery and on the cavernous body (vasodilator effect at a peripheric level) of the sildenafil nitrate salt and of the native sildenafil (citrate salt) is known. In the pharmacological experiment described in said application no information is given on the vascular tolerability of the compound in patients affected by various pathologies, for example cardiovascular pathologies. Indeed the vascular tolerability is a critical aspect if one considers that the medical speciality on the market which contains the sildenafil citrate salt is contraindicated, as above said, in cardiopathic patients.

The need was felt to have available drugs for sex dysfunctions not showing the aforesaid side effects of the citrate sildenafil.

The Applicant has unexpectedly and surprisingly found compounds able to solve this technical problem.

An object of the present invention is the systemic use, in particular oral and sublingual use, for the treatment of sex dysfunctions of one or more of the following classes of drugs:

A) organic or inorganic compounds or salts thereof, having general formula:

A—X₁—N(O)_{z}

as defined hereinunder,

C) Nitrate salts of compounds able to inhibit phosphodiesterases;

in the compounds of general formula:

A—X₁—N(O)_{z}

z is an integer and it is 1 or 2, preferably 2;

A=R(COXᵤ)ₜ and wherein t is an integer 0 or 1; u is 0 or 1;

X=O, NH, NR_{1c} wherein R_{1c} is a linear or branched C₁-C₁₀, alkyl;

X₁ is the following bivalent linking group:

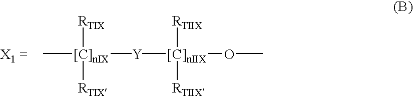

wherein:

nIX is an integer in the range 0-3, preferably 1;

nIIX is an integer in the range 1-3, preferably 1;

R_{TIX}, R_{TIX′}, R_{TIIX}, R_{TIIX′}, equal to or different from each other are H or linear or branched C₁-C₄ alkyl; preferably R_{TIX}, R_{TIX′}, R_{TIIX}, R_{TIIX′}, are H;

Y is a heterocyclic ring containing one or two nitrogen atoms, optionally one oxygen or sulphur atom, said saturated, unsaturated or aromatic ring, having 5 or 6 atoms.

R is selected from the following groups:

Group I) wherein t=1 and u=1

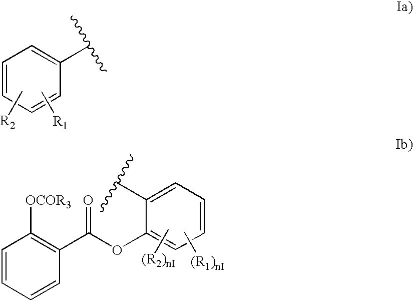

wherein:

R₁ is the OCOR₃ group; wherein R₃ is methyl, ethyl or linear or branched C₃-C₅ alkyl, or the residue of a heterocycle with only one ring having 5 or 6 atoms which can be aromatic, partially or totally hydrogenated, containing one or more heteroatoms independently selected from O, N and S;

R₂ is hydrogen, hydroxy, halogen, linear or branched when possible C₁-C₄ alkyl; a linear or branched when possible C₁-C₄ alkoxy; a linear or branched when possible C₁-C₄ perfluoroalkyl, for example trifluoromethyl; nitro, amino, mo- no- or di-(C₁₋₄) alkylamino;

nI is an integer 0 or 1;

preferably in the compounds of formula Ia) X is equal to O or NH, R₁ is acetoxy, preferably in ortho position with respect to —CO—, R₂ is hydrogen; preferably X₁ is the linking group (B) wherein R_{TIX}=R_{TIX′}=R_{TIIX}=R_{TIIX′}=H, n_{IX}=n_{IIX}=1;

Preferably in the compounds of formula Ib) R₃=CH₃, nI=0, X is equal to O, X₁ is as above defined for Ia); in this case Ib) is the residue of the acetylsalicylsalicylic acid;

Group II, wherein t=1, u=1

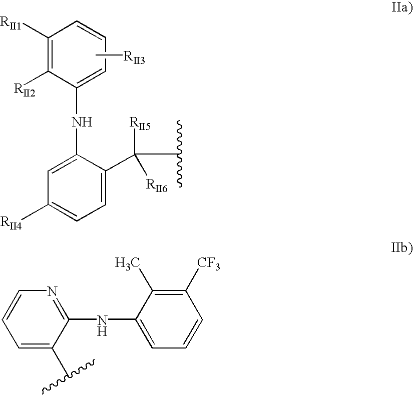

wherein:

R_{II5} is H, linear or branched when possible C₁-C₃ alkyl;

R_{II6} has the same meaning as R_{II5}, or when R_{II5} is H it can be benzyl;

R_{II1}, R_{II2} and R_{II3} can independently be hydrogen, linear or branched when possible C₁-C₆ alkyl, or linear or branched when possible C₁-C₆ alkoxy, or Cl, F, Br;

R_{II4} is R_{II1} or bromine;

the compounds wherein R_{II1}, R_{II4} are hydrogen and R_{II2} and R_{II3} are chlorine in ortho position with respect to NH are preferred;

R_{II5} R_{II6} are H, X is equal to O, and X₁ is as above defined for the compounds of formula Ia);

IIb) is the residue of the 2-[(2-methyl-3-(trifluoromethyl) phenyl[amino]-3-pyridincarboxylic] acid and when the —COOH group is present the compound is known as flunixin;

Group III) wherein t=1, u=1 and R is

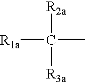

wherein:

R₂ₐ and R₃ₐ are H, linear or branched when possible, substituted or not, C₁-C₁₂ alkyl or allyl, with the proviso that if one of the two is allyl, the other is H; preferably R₂ₐ is H, C₁-C₄ alkyl, R₃ₐ is H;

R₁ₐ is selected from

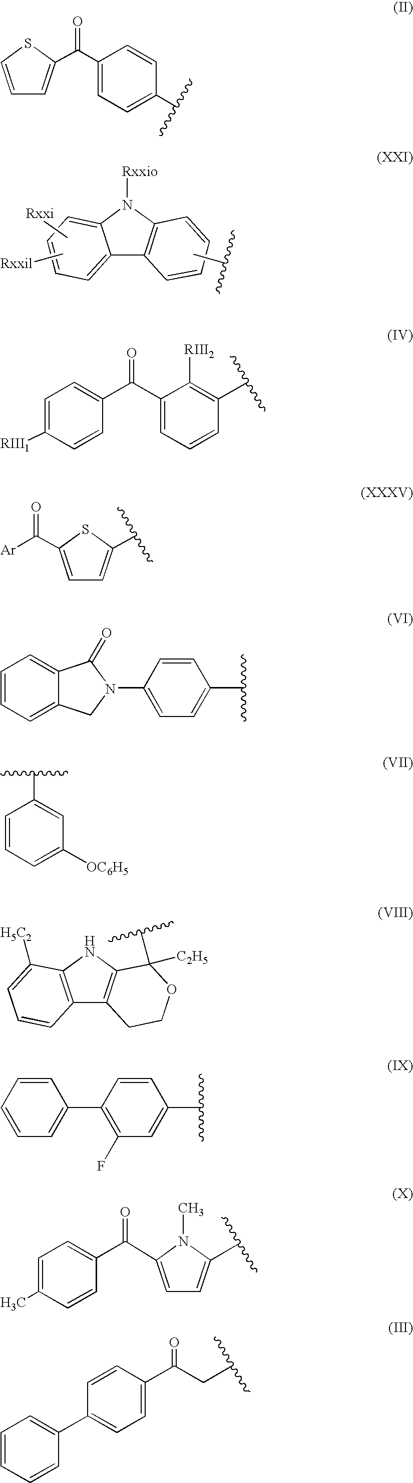

IIID) R₁ₐ corresponds to the following formulas:

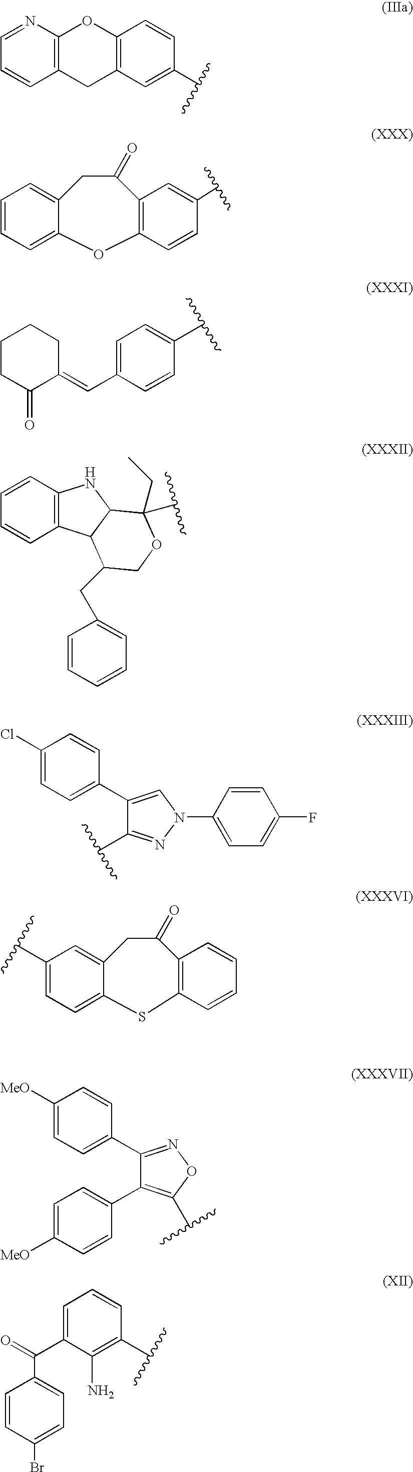

wherein the meanings are the following:

when R₁ₐ is as defined in formula (IV), Ketoprofen residue:

R_{III1} is H, SR_{III3} wherein R_{III3} contains from 1 to 4 carbon atoms, linear or branched when possible;

R_{III2} is H, hydroxy;

the compounds wherein R_{III1} and R_{III2} are H, R₃ₐ is H, and

R₂ₐ, is methyl, X=O, are preferred;

when R₁ₐ is as adefined in formula (XXI), carprofen residue:

Rₓₓᵢₒ is H, linear or branched when possible alkyl from 1 to 6 carbon atoms, C₁-C₆ alkoxycarbonyl linked to a C₁-C₆ alkyl, C₁-C₆ carboxyalkyl, C₁-C₂ alkanoyl, optionally substituted with halogens, benzyl or halobenzyl, benzoyl or halobenzoyl;

Rₓₓᵢ is H, halogen, hydroxy, CN, C₁-C₆ alkyl optionally containing OH groups, C₁-C₆ alkoxy, acetyl, benzyloxy, SRₓₓᵢ₂ wherein Rₓₓᵢ₂ is C₁-C₆ alkyl; C₁-C₃ perfluoroalkyl;

C₁-C₆ carboxyalkyl optionally containing OH groups, NO₂, amino; sulphamoyl, di-alkyl sulphamoyl with C₁-C₆ alkyl, or difluoroalkylsulphonyl with C₁-C₃ alkyl;

Rₓₓᵢ₁ is halogen, CN, C₁-C₆ alkyl containing one or more OH groups, C₁-C₆ alkoxy, acetyl, acetamido, benzyloxy, SR_{III3} being R_{III3} as above defined, C₁-C₃ perfluoroalkyl, hydroxy, C₁-C₆ carboxyalkyl, NO₂, amino, mono- or di-alkyl-amino C₁-C₆; sulphamoyl, di-alkyl sulphamoyl C₁-C₆, or di-fluoroalkylsulphamoyl as above defined; or Rₓₓᵢ together with Rₓₓᵢ₁ is a C₁-C₆ alkylen dioxy;

the compounds are preferred wherein Rₓₓᵢₒ is H, the linking group is in position 2, Rₓₓᵢ is H, Rₓₓᵢ₁ is chlorine and is in para position with respect to nitrogen;

R₃ₐ is H, R₂ₐ is methyl and X is O;

when R₁ₐ is as defined in formula (XXXV), tiaprofenic acid residue:

Ar is phenyl, hydroxyphenyl optionally mono- or polysubstituted with halogen, alkanoyl and alkoxy C₁-C₆, C₁-C₆ preferably C₁C₃ . trialkyl, cyclopentyl, cyclohexyl, cycloheptyl, heteroaryl, preferably thienyl, furyl optionally containing OH, pyridyl;

the preferred compounds of (XXXV) are those wherein Ar is phenyl, R₃ₐ is H, R₂ₐ is methyl and X is O;

when R₁ₐ is as defined in formula (II), suprofen residue, of which the preferred one has been indicated, wherein R₃ₐ is H, A₂ₐ is methyl and X=O, as described and obtained in U.S. Pat. No. 4,035,376 herein incorporated by reference;

when R₁ₐ is as defined in formula (VI), R is the residue of indoprofen when R₂ₐ=H and R₃ₐ=CH₃; of indobufen when R₂ₐ is equal to H and R₃ₐ=C₂H₅; X=O, as described and obtained according to U.S. Pat. No. 3,997,669 herein incorported by reference;

when R₁ₐ is as defined in formula (VIII), R is the etodolac residue when R₂ₐ=R₃ₐ=H and X=O, as described and obtained according to U.S. Pat. No. 3,843,681 herein incorporated by reference;

when R₁ₐ is as defined in formula (VII), R is the fenoprofen residue when R₃ₐ=H, R₂ₐ=CH₃ and X=O, as described and obtained according to U.S. Pat. No. 3,600,437 herein incorporated by reference;

when R₁ₐ is as defined in formula (III), R is the fenbufen residue when R₂ₐ=R₃ₐ=H and X=O, as described and obtained according to U.S. Pat. No. 3,784,701 herein incorporated by reference;

when R₁ₐ is as defined in formula (IX), R is the flurbiprofen residue when R₃ₐ=H, R₂ₐ=CH₃, X=O;

when R₁ₐ is as defined in formula (X) R is the tolmetin residue when R₂ₐ=R₃ₐ=H, X=O, as described and obtained according to FR 1,574,570 herein incorporated by reference;

In group IIID) R₁ₐ corresponds to the following formulas:

IIIa), when R₂ₐ=H and R₃ₐ=CH₃ the pranoprofen residue is obtained: α-methyl-5H-[1]benzopyran-[2,3-b]pyridin-7-acetic acid; the preferred compound has R₂ₐ=H, R₃ₐ=CH₃, u=1 and X=O:

(XXX), when R₂ₐ=H and R₃ₐ=CH₃, the bermoprofen residue is obtained: dibenz[b,f]oxepin-2-acetic acid; the preferred compound has R₂ₐ=H, R₃ₐ=CH₃, u=1 and X=O.

(XXXI) , when R₂ₐ=H and R₃ₐ=CH₃, R is the radical of the compound CS-670: 2-[4-(2-oxo-1-cyclohexyliden methyl) phenyl]propionic acid; the preferred compound has R₂ₐ=H, R₃ₐ=CH₃, u=1 and X=O;

(XXXII), when R₂ₐ=R₃ₐ=H, the Pemedolac residue is obtained; the preferred compound has R₂ₐ=R₃ₐ=H, u=1 and X=O;

(XXXIII), when R₂ₐ=R₃ₐ=H, the pirazolac residue is obtained: 4-(4-chlorophenyl)-1-(4-fluorophenyl)-3-pyrazolic acid derivatives;

The preferred compounds have R₂ₐ=R₃ₐ=H, u=1 and X=O;

(XXXVI), when R₂ₐ=H, R₃ₐ=CH₃, the zaltoprofen residue is obtained; when the residue is saturated with a hydroxyl or amino group, or with the carboxylic function the compounds are known as dibenzothiepine derivatives; the preferred compounds have R₂ₐ=H, R₃ₐ=CH₃,u=1 and X=O;

(XXXVII), when R₂ₐ=R₃ₐ=H the mofezolac residue is obtained: 3,4-di(p-methoxyphenyl)isoxazol-5-acetic acid when the residue is CH₂—COOH; the preferred compounds have R₂ₐ=R₃ₐ=H, t=1 and X=O;

(XII), when R₂ₐ=R₃ₐ=H the bromfenac residue is obtained: 2-amino-3-(4-bromobenzoyl)benzeneacetic acid; the preferred compounds have u=1, t=1, X=O, R₂ₐ=R₃ₐ=H; or t=0;

in group IV) wherein t=1, u=1, R is

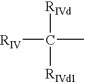

wherein:

R_{IVd} and R_{IVd1} are at least one H and the other a linear or branched when possible C₁-C₆ preferably C₁ and C₂ alkyl, or difluoroalkyl with the alkyl from 1 to 6 carbon atoms, C₁ is preferred, or R_{IVd} and R_{IVd1} form together a methylene group;

R_{IV} has the following meaning:

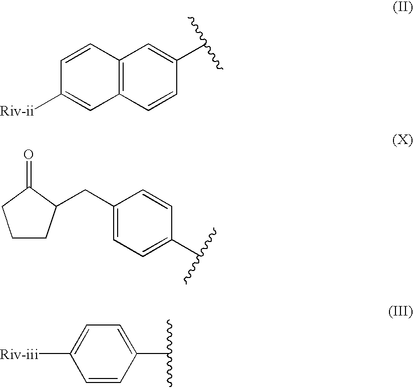

wherein the compounds of group IV) have the following meanings:

in formula (II)

Rᵢᵥ₋ᵢᵢ is C₁-C₆ alkyl, C₃-C₇ cycloalkyl, C₁-C₇ alkoxymethyl, C₁-C₃ trifluoroalkyl, vinyl, ethynyl, halogen, C₁-C₆ alkoxy, difluoroalkoxy, with C₁-C₇ alkyl, C₁-C₇ alkoxymethyloxy, alkylthiomethyloxy with C₁-C₇ alkyl, alkyl methylthio with C₁-C₇ alkyl, cyan, difluoromethylthio, phenyl- or phenylalkyl substituted with C₁-C₈ alkyl;

preferably Rᵢᵥ₋ᵢᵢ is CH₃O—, R_{IVd} is H and R_{IVd1} is CH₃ and it is known as naproxen residue;

X=O and X₁ is as above defined for Ia);

in formula (X), of which the loxoprofen residue, described in U.S. Pat. No. 4,161,538 herein incorporated by reference, has been indicated, the compounds wherein R_{IVd} is H and R_{IVd1} is CH₃, X=O and X₁ is as above defined for Ia) are preferred;

in formula (III):

Rᵢᵥ₋ᵢᵢᵢ is a C₂-C₅ alkyl, optionally branched when possible, C₂ and C₃ alkyloxy, allyloxy, phenoxy, phenylthio, cycloalkyl from 5 to 7 carbon atoms, optionally substituted in position 1 by a C₁-C₂ alkyl;

it is preferred the compound wherein Rᵢᵥ₋ᵢᵢᵢ is

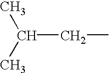

and R_{IVd}=H, R_{IVd1} is CH₃, compound known as ibuprofen residue; X=O and X₁ is as above defined for Ia);

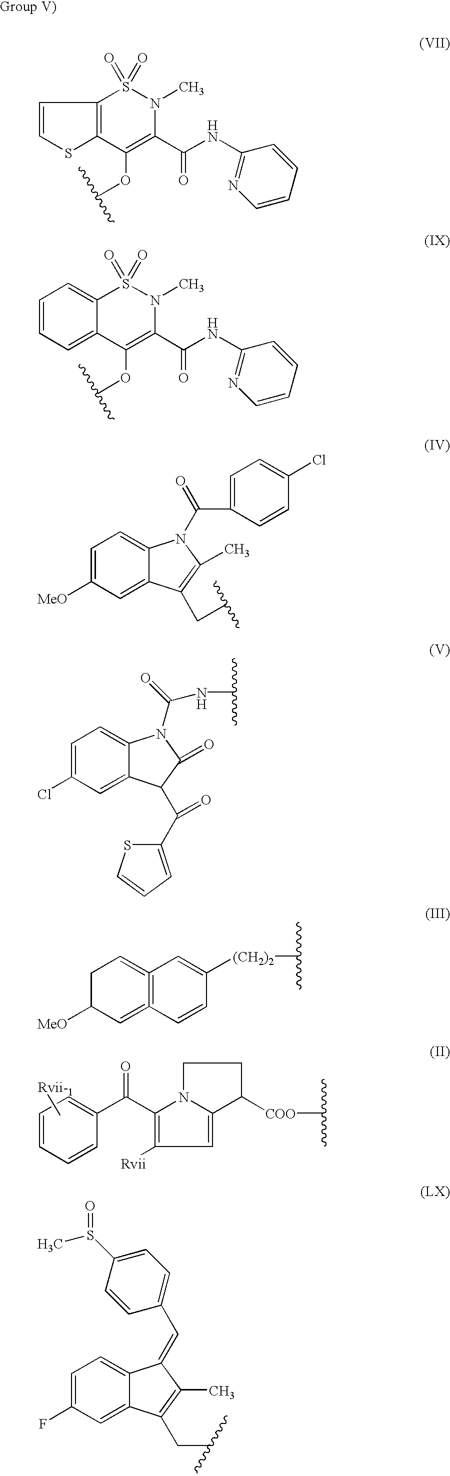

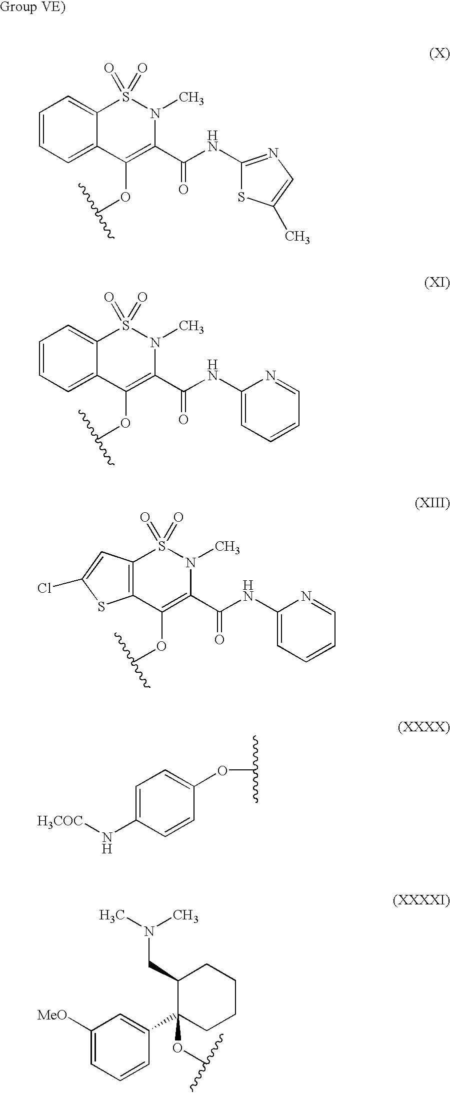

in group V), the compounds have the following meanings:

when R is formula (II),

Rᵥᵢᵢ is H or a linear or branched when possible C₁-C₄ alkyl;

Rᵥᵢᵢ₋₁ is Rᵥᵢᵢ, or a linear or branched when possible C₁-C₄ alkoxy; Cl, F, Br; the position of Rᵥᵢᵢ₋₁ being ortho, or meta, or para;

the residue of the known Ketorolac is preferred, wherein Rᵥᵢᵢ and Rᵥᵢᵢ₋₁ are H, and A=R (A being the group of the formula A—X—NO₂) and t=0;

when R is formula (V),

of which the residue of the known tenidap has been indicated, as described and obtained in U.S. Pat. No. 4,556,672 herein incorporated by reference;

in these compounds of formula (V) A=R and t=0,

when R is formula (VII),

of which the residue of the known tenoxicam has been indicated, A is RCO, t=1 u=0 or A is R and t=0, as described and obtained in DE 2,537,070 herein incorporated by reference;

when R is formula (IX),

wherein A=R and t=0, or A=RCO with t=1 and u=0, the residue of the known piroxicam has been indicated, as described and obtained in U.S. Pat. No. 3,591,584 herein incorporated by reference;

when R is formula (III)

wherein A=RCOO, t=1 and u=0 or 1; or t=0 and A=R, of which the residue of the known nabumetone has been indicated, as described and obtained in U.S. Pat. No. 4,061,779 herein incorporated by reference;

when R is formula (IV)

wherein A=RCOO, t=1 and u=1, of which the indomethacin residue has been indicated, as described and obtained in U.S. Pat. No. 3,161,654, herein incorporated by reference;

when R=formula (IX) and in (COXᵤ)ₜ u=t=1 and X is oxygen, the precursor compound is known as sulindac;

when R is formula (X), the X residue is known as meloxicam; the preferred compounds are those wherein A=RCO, t=1 and u=0;

when R is formula (XI) the residue is known as ampiroxicam when the termination is —CH(CH₃)OCOC₂H₅; the preferred compounds have A=RCO, t=1 and u=0;

when R is formula (XIII) and the valence is saturated with H, the residue derives from lornoxicam; the preferred compounds have A=RCO, t=1 and u=0;

when R is formula (XXXX) and the valence is saturated with H the compound known as paracetamol is obtained, as described and obtained in U.S. Pat. No. 2,998,450 herein incorporated by reference;

when R is formula (XXXXI) and the valence is saturated with H, the compound known as Tramadol is obtained, as described and obtained in U.S. Pat. No. 3,652,589;

the preferred compounds according to the present invention obtainable with the radicals corresponding to the formulas (XXXX) and (XXXXI) have A=RCO, t=1 and u=0.

Preferably Y is selected from the following:

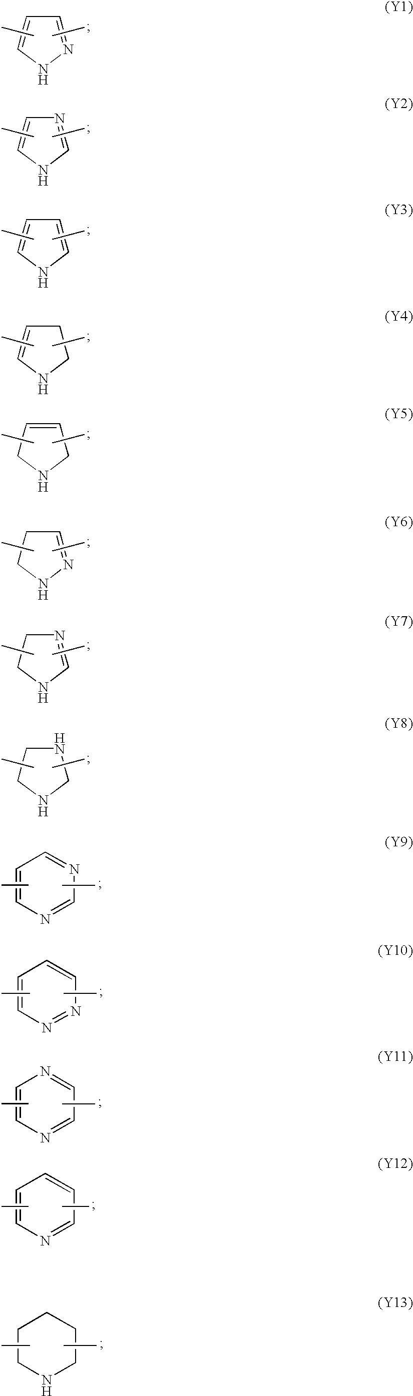

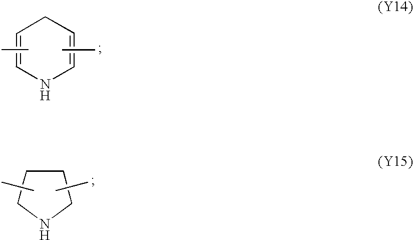

Preferably Y is an aromatic ring having 6 atoms, containing one nitrogen atom, said aromatic ring having the two free valences in position 2 and 6.

The preferred of Y is Y12 (pyridyl) substituted in position 2 and 6. The bonds can be also in a non symmetric position, for example Y12 (pyridyl) can be substituted also in position 2 and 3; Y1 (pyrazol) can be 3,5-disubstituted.

The X₁ precursors as defined by formula (B), wherein the free valence of the oxygen is saturated with H and the free valence of the end carbon is saturated either with a carboxylic or hydroxyl group, are commercially available compounds or they can be obtained by known methods of the prior art.

The compounds containing R of group I of the type Ia) are described in patent application WO 92/01668 wherein also the preparation methods are mentioned. This patent is herein incorporated by reference. The compounds of type Ib) are for example prepared by using the method indicated in The Merck Index, XI ed., 1989, pag. 16, No. 95 for the acetylsalicylsalicylic acid residue. The modifications of the compounds of formula Ib) can be obtained by using the processes mentioned in patent application WO 92/01668.

The compounds wherein R is of group II) are described in patent application WO 94/04484 and U.S. Pat. No. 3,558,690 wherein also the preparation methods are indicated. These patents are herein incorporated by reference.

The starting compound of IIb), when the valence is saturated with —COOH (flunixin), is obtained according to U.S. Pat. No. 3,337,570 and U.S. Pat. No. 3,689,653, both herein incorporated by reference. The compounds containing the substituents mentioned in the previous patents are equivalent to flunixin.

The compounds wherein R is of group III) are described and obtained by the processes mentioned in the following patents:

patent application PCT/EP/93 03193; for the compounds of formula (IV) see also U.S. Pat. No. 3,641,127; for the compounds of formula (XXI) see also U.S. Pat. No. 3,896,145; for the compounds of formula (IX) flurbiprofen residue see also U.S. Pat. No. 3,755,427; for the compounds of formula (II) see also U.S. Pat. No. 4,035,376; for the compounds of formula (VI) see also U.S. Pat. No. 3,997,669; for the compounds of formula (VIII) see also U.S. Pat. No. 3,843,681; for the compound of formula (VII) see also U.S. Pat. No. 3,600,437; for the compounds of formula (III) see also U.S. Pat. No. 3,784,701. All these mentioned patents are herein incorporated by reference.

The procedures for the preparation of the compounds of class IIID) are the following:

The residue IIIa) is obtained by preparing the acid compound according to U.S. Pat. No. 3,931,205, the valence is saturated with —CH(CH₃)—CCOH. The compounds containing the substituents mentioned in the previous patent are equivalent to pranoprofen. The residue (XXX) is prepared through the compound with the group —CH(CH₃)—COH (bermoprofen) according to U.S. Pat. No. 4,238,620 herein incorporated by reference. Other equivalent products are described in the above mentioned patent.

The residue (XXXI) is prepared by starting from the corresponding acid —CH(CH₃)—COOH according to U.S. Pat. No. 4,254,274. Equivalent compounds are described in the same patent.

The residue (XXXII) is prepared according to EP 238,226 herein incorporated by reference, when the valence is saturated with —CH₂—COOH. Equivalent products are reported in said patents as 1,3,4,9 tetrahydropyran [3,4-b] indol-1-acetic substituted acids.

The residue (XXXIII) is prepared from pirazolac and the valence is saturated with —CH₂—COOH, as indicated in EP 54,812 herein incorporated by reference. Equivalent products are described in said patent.

The residue (XXXVI) is prepared according to UK 2,035,311 herein incorporated by reference, by starting from zaltoprofen and having the —CH(CH₃)—COOH termination. Equivalent products are described in said patent.

The process for preparing the residue (XXXVII) is obtained by starting from mofezolac and it is prepared according to EP 26,928. Equivalent products are reported in the same patent.

The compounds wherein R is of group IV) are described in GB patent application 2,283,238, wherein also the preparation methods are indicated; this patent is herein incorporated by reference.

In group IV) the compounds can also be obtained:

for the compounds of formula (II) using U.S. Pat. No. 3,904,682; the compounds of formula (X) according to U.S. Pat. No. 4,161,538; the compounds of formula (III) according to U.S. Pat. No. 3,228,831. The herein mentioned patents are incorporated in the present application by reference.

In group V) the compounds can also be obtained: for the compounds of formula (II) using U.S. Pat. No. 4,089,969 herein incorporated by reference; the compounds of formula (V) can be obtained according to U.S. Pat. No. 4,556,672 herein incorporated by reference.

The residue (X) is prepared according to the German patent 2,756,113. Equivalent products are described in said patent.

The residue (XI) is prepared according to EP 147,177, herein incorporated by reference, starting from ampiroxicam having the termination —CH(CH₃)OCOOC₂H₅. Equivalent products are described in said patent.

The residue (XII) is prepared according to J. Med. Chem., vol. 27 No. 11, November 1984, Walsh et Al. “Antiinflammatory Agents. 3. Synthesis and Pharmacological Evaluation of 2-amino-3-benzoylphenylacetic Acid and Analogues”, herein incorporated by reference. Equivalent products are described in said publication.

The residue (XIII) is prepared starting from lornoxicam, wherein the valence is saturated with H. It is prepared according to GB 2,003,877. Equivalent products are described in said patent.

The residue (LX) in group V is prepared from Sulindac, obtained according to U.S. Pat. No. 3,654,349.

In general the connection between A and X₁ is, as seen, of ester or amidic type (NH or NR_{1C}, as defined in X) when R is of groups I, II, III, IV and V. For the formation of such connection all the synthesis routes well known for the formation of such bonds are usable.

The preparation of the compounds of formula A—X₁—N(O)_{z} with the linking group X₁ of formula (B) is described in published PCT application WO 00/51988 in the name of the Applicant, herein incorporated by reference.

The compounds inhibiting the phosphodiesterase C) salified with nitric acid are selected from the following: (C1) 1-[4-ethoxy-3-(6,7-dihydro-1-methyl-7-oxo-3-propyl-1H-pyra-zol[4,3-d]-pyrimidin-5-yl)-phenyl]sulphoyl[-4-methyl-piperazine (Sildenafil), (C2) 2-(2-propyloxyphenyl)-8-azapurin-6-one (Zaprinast), (C3) 2,6-bis-(diethanolamino)-4,8-dipiperidine pyrimido[5,4-d]-pyrimidine (dipyridamol), (C4) 6-chloro-4-(1,3-dioxaindan-5-yl)methylamino-2(4-carboxy-1-piperidinyl)-quinazoline, (C5) N-(phenylmethyl) -1-ethyl-1H-pyrazol-[3,4-b]-quinolin-4-amine, (C6) 1-(2-chlorobenzyl)-3-isobutyryl-2-propyl-6-aminocarbonyl-indol, (C7) 1-benzyl-6-choro-2-[1-[3-(imidazol-1-yl)propyl]indol-5-yl-amino carbonyl]benzimidazol, (C8) 2-(1-imidazolyl)-5-(phenyl)-4-(1,3-dioxaindan-5-yl)methyl aminopyrimidine, (C9) 6-ethynyl-4-(2-metoxyethyl)amino-2-(1-imidazolyl)quinazoline, (C10) 1-cyclo-pentyl-3-ethyl-6-(2-propoxyphenyl)pyrazol[3,4-d]pyrimidin-4-one, (C11) 1-cyclopentyl-3-ethyl-6-(4-methoxybenzyl)-pyrazol-[3,4-d]-pyrimidin-4-one, (C12) 1,3-dimethyl-6-(2-propoxy-5-methansulphonamidophenyl)-1,5-dihydropyrazol[3,4-d]-pyrimidin-4-one, (C13) (6R,12aR)-2,3,6,7,12,12a-hexahydro-2-methyl-6-(1,3-dioxan-5-yl)pyrazin [2′,1′:6,1]pyrido[3,4-b]indol-1,4-dione, (C14) 1-propyl-3-methyl-6-[2-propoxy-5-[(4′-methyl -1-pyrazinyl)sulphonamido]phenyl]-1,5-dihydropyrazol[3,4-d]pyrimidin-4-one, (C15) 3-(4-amino carbonyl-1-pi-peridinyl)-6-cyan-8-(3-chloro-4-methoxy-phthalazine, (C16) 2-(1-imidazolyl)-4-(1,3-dioxaindan-5-yl) methylamino-7,8-di-hydro-5H-thiopyran[3,2-d]pyrimidine, (C17) 1-Cyclopentyl-3-ethyl-6-(3-ethoxypyrid-4-yl)-1H-pyrazolo[3,4-d]pyrimidin-4-one, (C18) 1-[3-[1-[(4-Fluorophenyl)methyl]-7,8-dihydro-8-oxo-1H-imidazo[4,5-g]quinazolin-6-yl]-4-propoxyphenyl]carboxamide.

The pharmaceutical formulations usable for the specific use according to the present invention are those well known to the skilled in the art and which can be prepared according to the texts widely known in the prior art. See for exampe the volume “Remington's Pharmaceutical Sciences 15a Ed.”.

The dosages of the salts of the invention in their pharmaceutical compositions are equal, and generally lower than those of their precursors of the above mentioned classes, said salts generally being more effective and better tolerated.

The salts of the compounds A) and C) can be used as such, preferably in formulations administrable according to conventional administration routes of drugs. For example they can be administered by systemic route, for example by oral, sublingual route.

Surprisingly it has been found by the Applicant that the sildenafil nitrate has a power ratio, calculated as ratio between the myorelaxing effect on the cavernous body and the systemic pressure effect (see the data on the aorta reported in Table 1), clearly in favour of the myorelaxing effect. This shows that the sildenafil nitrate can be used for the impotence treatment also by cardiopathic people since the pressure effect (aorta) is very reduced.

For patients suffering from sex dysfunctions (male and female) it has been found that the salts of compounds A) and the nitrate salts of compounds C) for systemic use have a low pressure effect wherefore the power ratio, calculated as above, is improved with respect to the commercial sildenafil (citrate salt).

It has been unexpectedly found that the salts of the compounds of the invention can also be topically administered as such, preferably using the corresponding formulations containing them as active principles. This is a surprising fact since it is not said that a compound active by systemic route is active also by topical route. It has been unexpectedly found that also the salts of compounds C), different from nitrates, are active by topical route, as such or when administered carried in the above formulations.

Examples of organic salts of C) are oxalate, tartrate, maleate, succinate, citrate, glycinate, lysinate; examples of inorganic anions are nitrate, chloride, sulphate, phosphate.

The administration by topical route of compounds A) and of the salts of C), in particular of the phosphodiesterase inhibitors, was not predictable for the use according to the present invention, in particular for the treatment of the male impotence and of the female sex dysfunctions, since the myorelaxing action of said products is not direct but it takes place through the strengthening of the endogen mediator cGMP which is formed through the nitric oxide.

In particular, as regards the compositions for topical use, the salt amount of the compounds of classes A) and C) in the pharmaceutical form, for the predicted use according to the present invention, is in the range 0.5-10%, preferably 2-6%, as percentage by weight on the total weight of the composition. Said formulations for topical use can be in the form of salves, creams and gels and are prepared according to the techniques known to the skilled of the art, as described for example in the above mentioned volume.

The above compounds inhibiting the phosphodiesterases are synthesized as described in the following references (C1) G.B. 92480; (C2): DE 2162096; (C3): The Merck Index 12th Ed.; (C4) :WO 9422855; (C5): WO 9628159; (C6): WO 9632379; (C7): WO 9703070; (C8): U.S. Pat. No. 5,525,604; (C9): U.S. Pat. No. 5,436,233; (C10): WO 9628448; (C11): WO 9628429; (C12): EP 636626; (C13): WO 9519978; (C14): EP 636626; (C15): WO 9605176; (C16): EP 728759; (C17): U.S. Pat. No. 5,294,612; (C18): J. Med. Chem. 2000, 43, 1257-1263.

Constitutes a further object of the present invention nitrooxy derivatives of the following phosphodiesterase inhibitors:

(C2) 2-(2-propyloxyphenyl)-8-azapurin-6-one (zaprinast),

(C3) 2,6-bis-(diethanolamino)-4,8-dipiperidine pyrimido [5,4-d]-pyrimidine (dipyridamol),

(C4) 6-chloro-4-(1,3-dioxaindan-5-yl)methylamino-2(4-carboxy-1-piperidinyl)-quinazoline,

of formula:

A—X_{1A}—N(O)_{z}(IC)

Wherein A is as above defined, and

in the case of (C2) t=0 and R is the phenyloxy radical derived by substituting the ether group on the phenyl ring of Zaprinast with an hydroxy function (see Tetrahedron letters 1967 pages 4131 and following ones, Tetrahedron letters 1968 24 pages 2289 and following ones);

in the case of (C3) t=C and R is the alcoxy radical derived from the precursor;

in the case of (C4) t=u=1 and X is oxygen;

X_{1A} can have the meaning of X₁ above and also the following ones:

an alkylene group R′ wherein R′ is a C₁-C₂₀ linear or branched when possible, preferably having from 2 to 6 carbon atoms, optionally substituted with one or more of the following groups: —NHCOR₃, wherein R₃ is C₁-C₄ linear or branched alkyl, —NH₂, or OH

a cycloalkylene having from 5 to 7 carbon atoms, optionally substituted with side chain R′, R′ being as above, one or more carbon atoms of the cycloalkylene ring can optionally be substituted by heteroatoms;

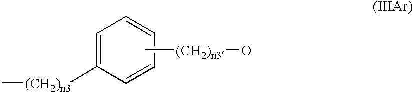

wherein n3 is an integer from 0 to 3 and n3′ is an integer from 1 to 3;

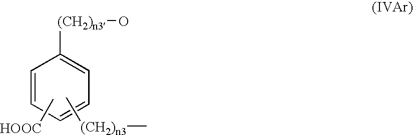

wherein n3 and n3′ have the above meaning;

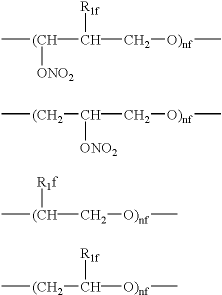

wherein R_{1f}=H, CH₃ and nf is an integer from 1 to 6, preferably from 1 to 4.

The compounds of formula (IC) as above defined can be prepared with known methods; when the bivalent linking bridge is of formula (B), the same methods above described apply. When the linking bridge have the other meanings the methods described in WO 95/30641.

The nitrate salts of the phosphodiesterase inhibitors can be prepared by known methods, for example as described in the patent application WO 99/67231; the other salts of compounds C) with anions different from nitrate are prepared by known methods of the prior art, such as for example described in patent application WO 96/28448.

The following Examples illustrate the invention but they do not limit the scope thereof.

### EXAMPLE 1

### Preparation of a Formulation for Topical Use Containing as Active Principle the 2(Acetyloxy)Benzoic Acid 6-(Nitroxy-methyl)-2-methylpyridyl Ester Hydrochloride (OCX 4050)

The compound is prepared according to Example 1 of patent application PCT/EP 00/01454.

Components of the formulation for topical use:

| | | |
|---|---|---|
| NCX 4050 | 4.2 | g |
| white vaseline | 24 | g |
| cetostearyl alcohol | 9.5 | g |
| polyoxyethylene (60 OE) sorbitan | 4.8 | g |
| monostearate (Polysorbate ® 60) | | |
| glycerine | 9.5 | g |
| purified water | 48 | g |
| total | 100 | g |

Preparation of the Formulation

In a weighed vessel the white vaseline (24 g) and the cetostearyl alcohol (9.5 g) are melted. To the melted mass (70° C.) a solution previously obtained by dissolving NCX 4050 (4.2 g), polysorbate® 60 (4.8 g) and glycerine (9.5 g) in fresh-boiled purified water is added under stirring. At the end of the addition one continues to stir until complete cooling of the mass and at last it is determined by weighing the evaporated water amount, which is added to the formulation until obtaining the required total weight (100 g).

### PHARMACOLOGICAL EXAMPLES

### EXAMPLE F1

The relaxing effect of the tested drugs on carvernous body tissues has been evaluated with experiments in vitro as a measure of the inhibiting action on the impotence, and on aorta tissues as expression of the undesired hypotensive effect.

Preparation of Tissues

White New Zealand rabbits were sacrificed, cavernous body and aorta specimens were taken and suitably prepared for the determination of the myorelaxing activity in vitro, according to the procedure described by J. Jeremy (Br. J. Urology 79,958-63,1997).

The tissues were precontracted with phenylephrine (10 μM) and the relaxation was determined in the presence of the compounds object of the invention.

The compounds examined in this test are reported in Table 1. The 2-(acetyloxy)benzoic acid 6-(nitroxy methyl)-2-methylpyridyl ester hydrochloride (NCX 4050) is prepared as described in patent application PCT/EP 00/01454 (Ex. 1) , the sildenafil nitrate has been prepared as described in patent application WO 99/67231 (Ex. 3). The products used in the experiment were dissolved in dimethylsulphoxide, except sodium nitroprussiate which was dissolved in distilled water.

The data of the Table show that the products of the invention are more effective than the reference substances in relaxing the cavernous body, and induce a lower vasorelaxing effect on the aorta.

### EXAMPLE F2

The effect of the sildenafil citrate and sildenafil nitrate on the aorta relaxation was evaluated with an experiment in vitro in the presence of a conventional NO-donor (sodium nitroprussiate). Under these conditions it is known that the sildenafil citrate causes hypotension.

The experiment was carried out as described in the previous Example, by using aorta tissues taken from white New Zealand rabbits. The tissue strips are treated first with sodium nitroprussiate 10⁻⁷ M, then a part of the strips was treated with sildenafil citrate 10⁻⁷ M and another part with sildenafil nitrate 10⁻⁷ M.

The results of the experiment are reported in Table 2 and are expressed as percentage of the aorta relaxation with respect to the initial treatment with sodium nitroprussiate and they show that the sildenafil nitrate causes a lower strengthening of the relaxing effect induced by sodium nitroprussiate compared with the sildenafil citrate. Therefore the sildenafil nitrate is less hypotensive than the sildenafil citrate.

TABLE 1
Experiment in vitro on the myorelaxing effect of the
cavernous body and of aorta of the following
compounds NCX 4050, sildenafil nitrate,
sildenafil citrate and sodium nitroprussiate as a
comparison.

| | Concentration | Cavernous body | Aorta | |
|---|---|---|---|---|
| Treatment | (M) | % relaxation | % | Power ratio |
| NCX 4050 | 10-6 | 80 | 80 | 1 |
| Sodium | 10-6 | 50 | 100 | 0.5 |
| Nitroprussiate | 10-6 | 100 | 20 | 5 |
| Sildenafil | | | | |
| Nitrate | | | | |
| Sildenafil | 3 × 10-5 | 50 | 75 | 0.66 |
| Citrate | | | | |

TABLE 2
Experiment in vitro on the myorelaxing effect on aorta tissues
pretreated with sodium nitroprussiate and then treated,
respectively, with sildenafil nitrate and sildenafil citrate

| | Concentration | Aorta relaxation |
|---|---|---|
| Treatment | (M) | (%) |
| Sodium | 10⁻⁷ | 100 |
| Nitroprussiate | | |
| Sildenafil | 10⁻⁷ | 120 |
| Nitrate | | |
| Sildenafil | 10⁻⁷ | 170 |
| Citrate | | |

1. Use for the treatment of sex dysfunctions of one or more of the following classes of drugs:
A) salified and non salified nitric oxide donor drugs, of formula
A—X₁—N(O)_{z}
wherein the meaning of the terms appearing in the formula is as defined hereunder;
C) nitrate salts of compounds inhibiting phospho-diesterases;
in the compounds of general formula:
A—X₁—N(O)_{z}
z is an integer and is 1 or 2, preferably 2;
A=R(COXᵤ)ₜ and wherein t is an integer 0 or 1; u is 0 or 1;
X=O, NH, NR_{1c} wherein R_{1c} is a linear or branched C₁-C₁₀ alkyl;
X₁ is the following bivalent linking group: 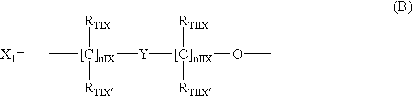
wherein:
nIX is an integer in the range 0-3;
nIIX is an integer in the range 1-3;
R_{TIX}, R_{TIX′}, R_{TIIX}, R_{TIIX′} equal to or different from each other are H or a linear or branched C₁-C₄ alkyl;
Y is an heterocyclic ring containing one or two nitrogen atoms, optionally one oxygen or sulphur atom, said saturated, unsaturated or aromatic ring having 5 or 6 atoms;
R of the radical A of formula A—X₁—N(O)_{z} is selected from the following groups:
Group I) wherein t=1 and u=1 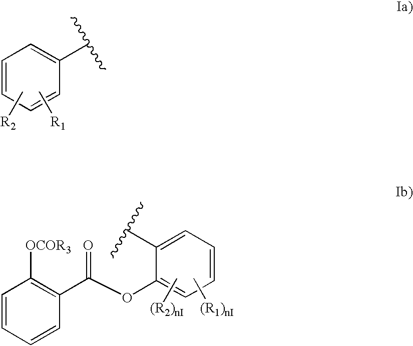
wherein:
R₁ is the OCOR₃ group; wherein R₃ is methyl, ethyl or a linear or branched C₃-C₅ alkyl, or the residue of an heterocycle having only one ring having 5 or 6 atoms which can be aromatic, partially or totally hydrogenated, containing one or more hetero-atoms independently selected from O, N and S;
R₂ is hydrogen, hydroxy, halogen, linear or branched C₁-C₄ alkyl, linear or branched C₁-C₄ alkoxy; a linear or branched C₁-C₄ perfluoroalkyl, for example trifluoromethyl; nitro, amino, mono- or di-(C₁₋₄) alkylamino;
nI is an integer 0 or 1;
group II) wherein t=1, u=1 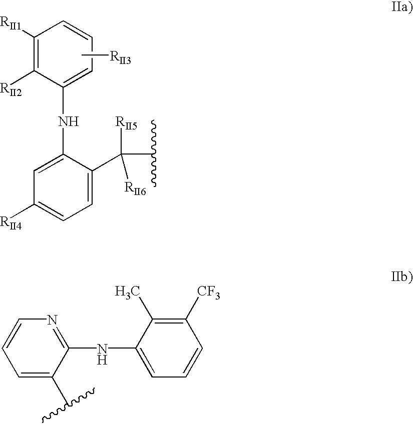
wherein:
R_{II5} is H, linear or branched when possible C₁-C₃ alkyl;
R_{II6} has the same meaning as R_{II5}, or when R_{II5} is H it can be benzyl;
R_{II1}, R_{II2} and R_{II3} can independently be hydrogen, linear or branched when possible C₁-C₆ alkyl, or linear or branched when possible C₁-C₆ alkoxy, or Cl, F, Br;
R_{II4} is R_{II1}, or bromine;
IIb) is the residue of the 2-[(2-methyl-3-(trifluoromethyl)phenyl]amino]-3-pyridincarboxylic]acid and when the —COOH group is present it is known as flunixin;
group III) wherein t=1, u=1 and R is 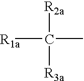
wherein:
R₂ₐ and R₃ₐ are H, linear or branched when possible, substituted or not, C₁-C₁₂ alkyl or allyl, with the proviso that if one of the two is allyl the other is H;
preferably R₂ₐ is H, C₁-C₄ alkyl, R₃ₐ is H;
R₁ₐ is selected from 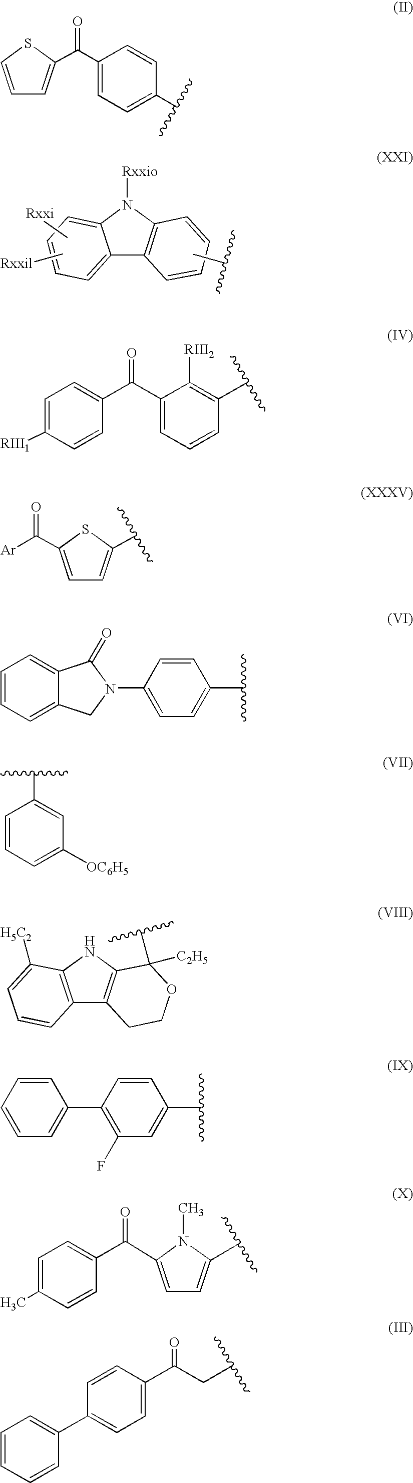
IIID) R₁ₐ corresponds to the following formulas: 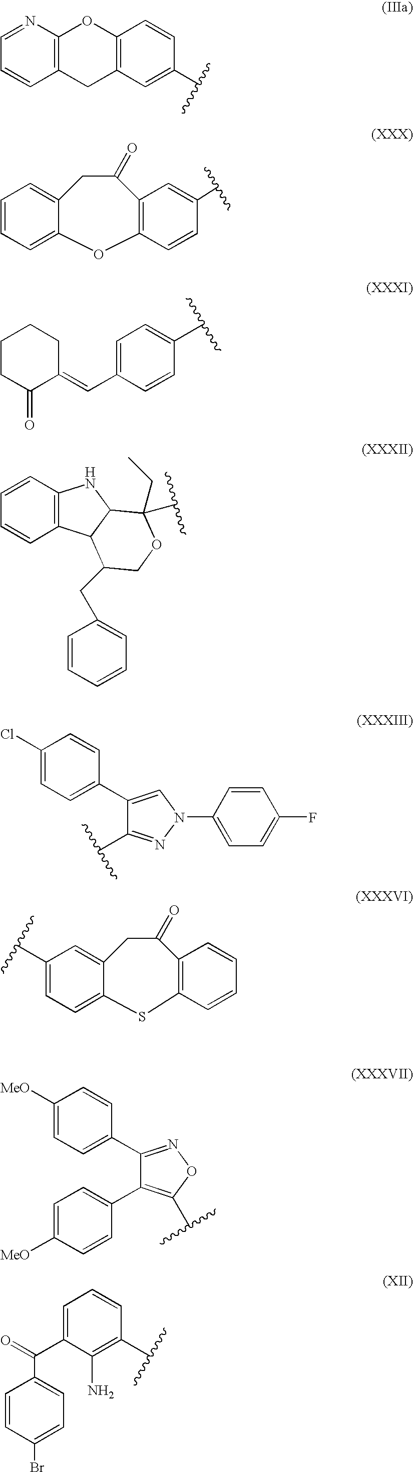
wherein the meanings are the following:
when R₁ₐ is as defined in formula (IV), Ketoprofen residue: R_{III1} is H, SR_{III3} wherein R_{III3} contains from 1 to 4 carbon atoms, linear or branched when possible; R_{III2} is H, hydroxy;
when R₁ₐ is as defined in formula (XXI), carprofen residue: Rₓₓᵢₒ is H, linear or branched when possible alkyl from 1 to 6 carbon atoms, C₁-C₆ alkoxycarbonyl linked to a C₁-C₆ alkyl, C₁-C₆ carboxyalkyl, C₁-C₆ alkanoyl, optionally substituted with halogens, benzyl or halobenzyl, benzoyl or halobenzoyl;
Rₓₓᵢ is H, halogen, hydroxy, CN, C₁-C₆ alkyl optionally containing OH groups, C₁-C₆ alkoxy, acetyl, benzyloxy, SRₓₓᵢ₂, wherein Rₓₓᵢ₂ is C₁-C₆ alkyl;
C₁-C₃ perfluoroalkyl; C₁-C₆ carboxyalkyl optionally containing OH groups, NO₂, amino; sulphamoyl, di-alkyl sulphamoyl with C₁-C₆ alkyl or difluoroalkyl-sulphonyl with C₁-C₃ alkyl;
Rₓₓᵢ₁ is halogen, CN, C₁-C₆ alkyl containing one or more OH groups, C₁-C₆ alkoxy, acetyl, acetamido, benzyloxy, SR_{III3} being R_{III3} as above defined, C₁-C₃ perfluoroalkyl, hydroxy, C₁-C₆ carboxyalkyl, NO₂, amino, mono- or di-alkyl-amino C₁-C₆; sulphamoyl, di-alkyl sulphamoyl C₁-C₆, or di-fluoroalkylsul-phamoyl as above defined; or Rₓₓᵢ, together with Rₓₓᵢ₁ is an alkylen dioxy C₁-C₆;
when R₁ₐ is as defined in formula (XXXV) tiaprofenic acid residue:
Ar is phenyl, hydroxyphenyl optionally mono or polysubstituted with halogen, alkanoyl and alkoxy C₁-C₆, trialkyl C₁-C₆, preferably C₁-C₃, cyclopentyl, cyclohexyl, cycloheptyl, heteroaryl, preferably tienyl, furyl optionally containing OH, pyridyl;
when R₁ₐ is as defined in formula (II), suprofen residue, wherein R₃ₐ is H, R₂ₐ is methyl and X=O;
when R₁ₐ is as defined in formula (VI), R is the residue of indoprofen when R₂ₐ=H and R₃ₐ=CH₃; of indobufen when R₂ₐ is equal to H and R₃ₐ=C₂H₅; X=O;
when R₁ₐ is as defined in formula (VIII) R is the etodolac residue when R₂ₐ=R₃ₐ=H and X=O;
when R₁ₐ is as defined in formula (VII), R is the fenoprofen residue when R₃ₐ=H, R₂ₐ=CH₃ and X=O;
when R₁ₐ is as defined in formula (III), R is the fenbufen residue when R₂ₐ=R₃ₐ=H and X=O;
when R₁ₐ is as defined in formula (IX), R is the flurbiprofen residue when R₃ₐ =H, R₂ₐ=CH₃, X=O;
when R₁ₐ is as defined in formula (X) R is the tolmetin residue when , R₂ₐ=R₃ₐ=H, X=O;
in group IIID) R₁ₐ corresponds to the following formulas:
IIIa), when R₂ₐ=H and R₃ₐ=CH₃ the pranoprofen residue is obtained: α-methyl-5H-[1]benzopyran-[2,3-b]pyridin-7-acetic acid; the preferred compound has R₂ₐ=H, R₃ₐ=CH₃, u=1 and X=O:
(XXX), when R₂ₐ=H and R₃ₐ=CH₃ the bermoprofen residue is obtained: dibenz[b,f]oxepin-2-acetic acid;
(XXXI), when R₂ₐ=H and R₃ₐ=CH₃, R is the radical of the CS-670 compound: 2-[4-(2-oxo-1-cyclo-hexyliden methyl) phenyl]propionic acid;
(XXXII), when R₂ₐ=R₃ₐ=H the Pemedolac residue is obtained;
(XXXIII), when R₂ₐ=R₃ₐ=H the pirazolac residue is obtained: 4-(4-chlorophenyl)-1-(4-fluoro phenyl)-3-pyrazolic acid;
(XXXVI), when R₂ₐ=H, R₃ₐ=CH₃ the zaltoprofen residue is obtained; when the residue is saturated with an hydroxyl or amino group, or with the carboxylic function the compounds are known as dibenzothiepine derivatives;
(XXXVII), when R₂ₐ=R₃ₐ=H the mofezolac residue is obtained: 3,4-di(p-methoxyphenyl)isoxazol-5-acetic acid;
(XII), when R₂ₐ=R₃ₐ=H the bromfenac residue is obtained: 2-amino-3-(4-bromobenzoyl)benzeneacetic acid;
in group IV) wherein t=1, u=1, R is 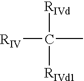
wherein:
R_{IVd} and R_{IVd1} are at least one H and the other a linear or branched C₁-C₆, preferably C₁ and C₂ alkyl, or difluo-roalkyl with the alkyl from 1 to 6 carbon atoms, C₁ is preferred, or R_{IVd} and R_{IVd1} form together a methylene group;
R_{IV} has the following meaning: 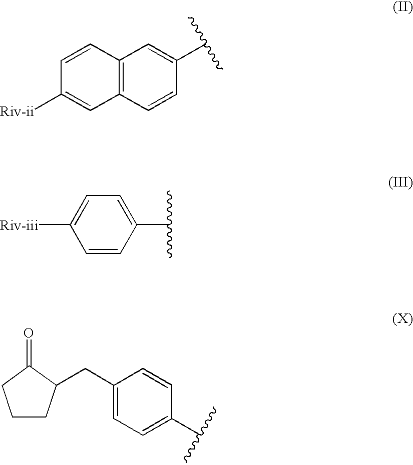
wherein the compounds of group IV) have the following meanings:
in formula (II):
Rᵢᵥ₋ᵢᵢ is C₁-C₆ alkyl, C₃-C₇ cycloalkyl, C₁-C₇ alkoxy-methyl, C₁-C₃ trifluoroalkyl, vinyl, ethynyl, halogen, C₁-C₆ alkoxy, difluoroalkoxy, with C₁-C₇ alkyl, C₁-C₇ alkoxymethyloxy, alkylthio methyloxy with C₁-C₇ alkyl, alkyl methylthio with C₁-C₇ alkyl, cyano, difluoromethylthio, phenyl- or phenylalkyl substituted with C₁-C₈ alkyl.
formula (X) loxoprofen residue;
in formula (III):
Rᵢᵥ₋ᵢᵢᵢ is a C₂-C₅ alkyl, optionally branched when possible, C₂ and C₃ alkyloxy, allyloxy, phenoxy, phenylthio, cycloalkyl from 5 to 7 carbon atoms, optionally substituted in position 1 by a C₁-C₂ alkyl; 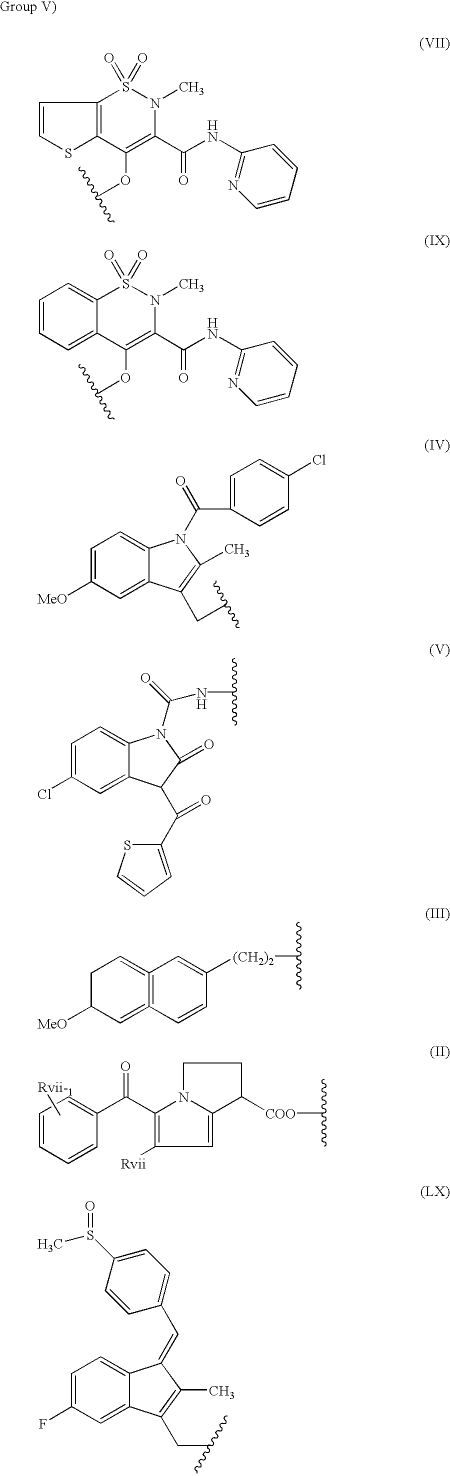 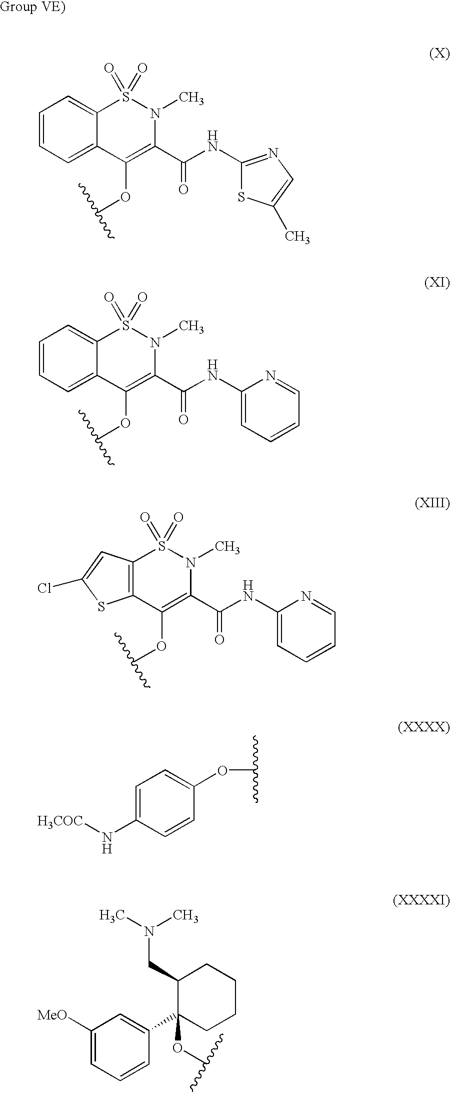
In group V):
when R is formula (II), Rᵥᵢᵢ is H or a linear or branched C₁-C₄ alkyl;
Rᵥᵢᵢ₋₁ is Rᵥᵢᵢ, or a linear or branched C₁-C₄ alkoxy;
Cl, F, Br; the position of Rᵥᵢᵢ₋₁ being ortho, or metha, or para;
when R is formula (V),
of which the residue of the known tenidap has been indicated;
When R is formula (V) A=R and t=O,
when R is formula (VII), A is RCO, t=1 u=0 or A is R and t=0;
when R is formula (IX), A=R and t=0, or A=RCO with t=1 and u=0;
when R is formula (III) A=RCOO, t=1 and u=0 or 1; or t=0 and A=R;
when R is formula (IV) A=RCOO, t=1 and u=1;
when R is formula (IX) and in (COXᵤ)ₜ u=t=1 and X is oxygen, the precursor compound is sulindac;
when R is formula (X) it is the meloxicam residue;
when R is formula (XI) the residue is known as ampiroxicam when the termination is —CH(CH₃)OCOC₂H₅;
when R is formula (XIII) and the valence is saturated with H, the residue derives from lornoxicam;
when R is formula (XXXX) and the valence is saturated with H the compound is known as paracetamol;
when R is formula (XXXXI) and the valence is saturated with H the compound is known as tramadol.

2. Use according to claim 1, wherein Y is selected from the following:

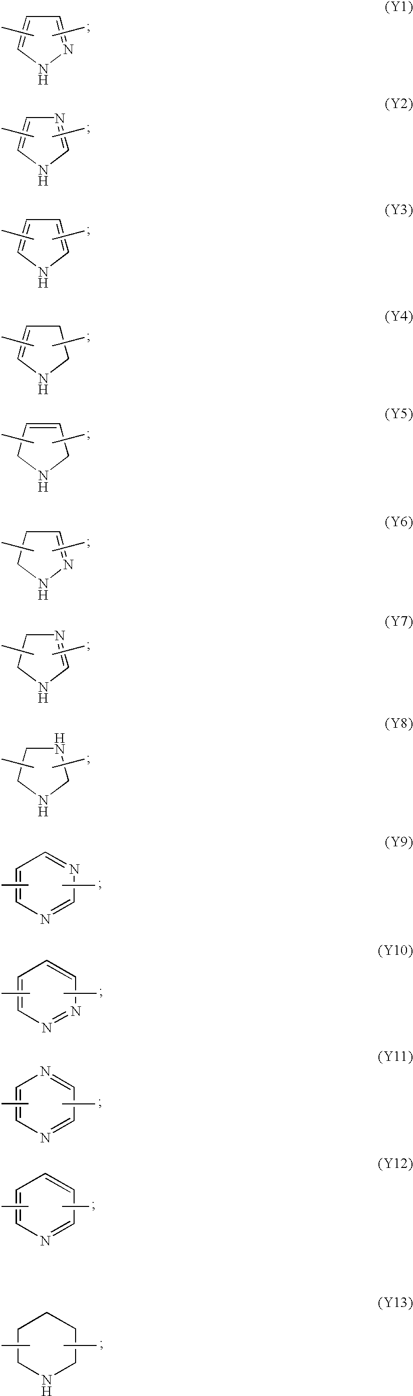

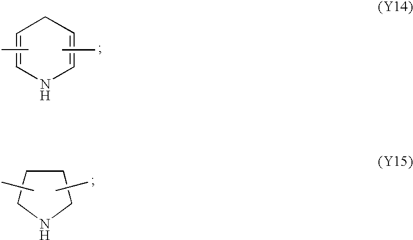

3. Use according to claim 2, wherein Y is Y12 (pyridyl) substituted in position 2 and 6.

4. Use according to claims 1-3, wherein in the compounds A) of formula A—X₁—N(O)_{z} z is 2 and nIX and nIIX in formula (B) of X₁ are integers equal to 1 and R_{TIX}, R_{TIX′}, R_{TIIX}, R_{TIIX′} are equal to H.

5. Use according to claims 1-4, wherein in the compounds of formula A) A—X₁—N(O)_{z} R, X, u and t of formula A=R(COXᵤ)ₜ, and Y in formula (B) of X₁, take the following meanings:
when R is selected from the group I), in the compounds of formula Ia) X is equal to O or NH, R₁ is acetoxy, preferably in ortho position with respect to —CO—, R₂ is hydrogen; in X₁ R_{TIX}=R_{TIX′}=R_{TIIX′}=H,
n_{IX}=n_{IIX}=1 and Y is an aromatic ring having 6 atoms, containing one nitrogen atom, said aromatic ring having the two free valences in position 2 and 6; in the compounds of formula Ib) R₃=CH₃, nI=0, X is equal to O, X₁ is as above defined for Ia); in this case Ib) is the residue of the acetylsa-licylsalicylic acid;
when R is selected in group II) in formula IIa R_{II1}, R_{II4} are hydrogen and R_{II2} and R_{II3} are chlorine in ortho position with respect to NH; R_{II5} and R_{II6} are H, X is equal to O, and X₁ is as above defined for the compounds of formula Ia);
when R is selected in group III),
when R₁ₐ is as defined in formula (IV) R_{III1} and R_{III2} are H, R₃ₐ is H, and R₂ₐ is methyl, X=O;
when R₁ₐ is as defined in formula (XXI) Rₓₓᵢₒ is H, the linking group is in position 2, Rₓₓᵢ is H, Rₓₓᵢ₁ is chlorine and it is in para position with respect to nitrogen;
when R₁ₐ is as defined in formula (XXXV) Ar is phenyl, R₃ₐ is H, R₂ₐ is methyl and X is O; R₃ₐ is H, R₂ₐ is methyl and X is O;
when R₁ₐ is as defined in formula IIIa), R₂ₐ=H, R₃ₐ=CH₃, u=1 and X=O;
when R₁ₐ is as defined in formula (XXX) R₂ₐ=H, R₃ₐ=CH₃, u=1 and X=O;
when R₁ₐ is as defined in formula (XXXI), R₂ₐ=H, R₃ₐ=CH₃, u=1 and X=O;
when R₁ₐ is as defined in formula (XXXII), R₂ₐ=R₃ₐ=H, u=1 and X=O;
when R₁ₐ is as defined in formula (XXXIII), R₂ₐ=R₃ₐ=H, u=1 and X=O;
when R₁ₐ is as defined in formula (XXXVI), R₂ₐ=H, R₃ₐ=CH₃, u=1 and X=O;
when R₁ₐ is as defined in formula (XXXVII), R₂ₐ=R₃ₐ=H, t=1 and X=O; when R₁ₐ is as defined in formula (XII) , R₂ₐ=R₃ₐ=H, u=1, t=1, X=O, R₂ₐ=R₃ₐ=H; or t=0;
when R is selected in group IV),
when R_{IV} is formula (II), Rᵢᵥ₋ᵢᵢ=CH₃O—, R_{IVd}=H and R_{IVd1}=CH₃, X=O and X₁ is as above defined for Ia);
when R_{IV} is formula (X), R_{IVd}=H, R_{IVd1}=CH₃, X=O and X₁ is as above defined for Ia);
when R_{IV} is formula (III), Rᵢᵥ₋ᵢᵢᵢ is 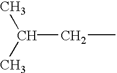
and R_{IVd}=H, R_{IVd1}, is CH₃, X=O and X₁ is as above defined for Ia);
when R is selected in group V,
when R is formula (II) , Rᵥᵢᵢ and Rᵥᵢᵢ₋₁ are H, and A=R;
when R is formula (X), A=RCO, t=1 and u=0;
when R is formula (XI), A=RCO, t=1 and u=0;
when R is formula (XIII), A=RCO, t=1 and u=0;
when R corresponds to formula (XXXX) or (XXXXI), A=RCO, t=1 and u=0.
Use according to claims 1-5, wherein the nitrate salts of the compounds inhibiting the phosphodiesterase are selected from the following: (C1) 1-[4-ethoxy-3-(6,7-dihydro-1-methyl-7-oxo-3-propyl-1H-pyra-zol[4,3-d]-pyrimidin-5-yl)-phenyl]sulphoyl]-4-methyl-piperazine (Sildena-fil), (C2) 2-(2-propyloxyphenyl)-8-azapurin-6-one (Zapri-nast), (C3) 2,6-bis-(diethanolamino)-4,8-dipiperidine py-rimido[5,4-d]-pyrimidine (dipyridamol), (C4) 6-chloro-4-(1,3-dioxaindan-5-yl)methylamino-2(4-carboxy-1-pyperidi-nyl)-quinazoline, (C5) N-(phenyl methyl)-1-ethyl-1H-pyra-zol-[3,4-b]-quinolin-4-amine, (C6) 1-(2-chlorobenzyl)-3-isobutyryl-2-propyl-6-amino carbonyl-indol, (C7) 1-benzyl-6-chloro-2-[1-[3-(imidazol-1-yl)propyl]indol-5-yl-amino carbonyl]benzimidazol, (C8) 2-(1-imidazolyl)-5-(phenyl)-4-(1,3-dioxaindan-5-yl)methyl aminopyrimidine, (C9) 6-ethynyl-4-(2-methoxyethyl)amino-2-(1-imidazolyl)quinazo-line, (C10) 1-cyclopentyl-3-ethyl-6-(2-propoxyphenyl) py-razol[3,4-d]pyrimidin-4-one, (C11) 1-cyclopentyl-3-ethyl-6-(4-methoxybenzyl)-pyrazol-[3,4-d]-pyrimidin-4-one, (C12) 1,3-dimethyl-6-(2-propoxy-5-methansulphonamidophenyl)-1,5-dihydro pyrazol[3,4-d]-pyrimidin-4-one, (C13) (6R,12aR)-2,3,6,7,12,12a-hexahydro-2-methyl-6-(1,3-dioxan-5-yl)pyrazine[2′,1′:6,1]pyrido[3,4-b]indol-1,4-dione, (C14) 1-propyl-3-methyl-6-[2-propoxy-5-[(4′-me-thyl-1-pyrazinyl)sul-phonamido]phenyl]-1,5-dihydropyra-zol[3,4-d]pyrimidin-4-one, (C15) 3-(4-amino carbonyl-1-piperidinyl)-6-cyan-8-(3-chloro-4-methoxy-phthalazine, (C16) 2-(1-imidazolyl)-4-(1,3-dioxaindan-5-yl)methylamino-7,8-dihydro-5H-thiopyran[3,2-d]pyrimidine, (C17) 1-Cyclopentyl-3-ethyl-6-(3-ethoxypyrid-4-yl)-1H-pyrazolo[3,4-d]pyrimidin-4-one, (C18) 1-[3-[1-[(4-Fluorophenyl)methyl]-7,8-dihydro-8-oxo-1H-imidazo[4,5-g]quinazolin-6-yl]-4-propoxyphenyl]carboxamide.

7. Use according to claims 1-6, obtained by the pharmaceutical formulations containing one or more salts of classes A) and C).

8. Use according to claim 7, wherein said formulations are administrable by oral and sublingual route.

9. Use according to claims 1-7 wherein said formulations are for topical use and comprise as active principles also the salts of compounds C) different from nitrates.

10. Use according to claim 9, wherein the organic anions of said salts of compounds C) different from nitrates are selected from oxalate, tartrate, maleate, succinate, citrate, glycinate, lysinate; and the inorganic ones are selected from chloride, sulphate, phosphate.

11. Use according to claims 9-10, wherein the formulations for topical use comprise an active principle amount in the range 0.5 and 10% by weight.
